(19) United States
(12) Patent Application Publication
(10) Pub. No.: US 2009/0054338 A1
(43) Pub. Date: Feb. 26, 2009

(54) Synergisms between repartitioning and immunomodulating compounds

(76) Inventor: A.K. Gunnar Aberg, Sarasota, FL (US)

Correspondence Address: Nields & Lemack, 176 E. Main Street, Suite #5, Westboro, MA, 01581, US

(21) Appl. No.: 12/221,747

(22) Filed: Aug. 6, 2008

Related U.S. Application Data
(60) Provisional application No. 60/963,607, filed on Aug. 6, 2007.
(60) Provisional application No. 60/965,127, filed on Aug. 18, 2007.

(51) Int. Cl. A61K 38/17; A61K 31/716

(52) U.S. Cl. 514/12; 514/54

(57) ABSTRACT

Combination therapies of adrenergic beta-receptor agonists and beta-glucans are described.

This patent application claims priority of provisional application Ser. No. 60/963,607 filed on Aug. 6, 2007 and provisional application Ser. No. 60/965,127, filed on Aug. 18, 2007, the disclosures of both provisional applications are hereby incorporated by reference.

### FIELD OF THE INVENTION

The present invention relates to unexpected synergistic effects by co-administration of an adrenergic beta-receptor agonist and an immunomodulating beta-glucan.

### BACKGROUND OF THE INVENTION

There are significant problems facing the livestock industry as animal growth is slow, while morbidity and mortality are high in the cattle- and pig-farms and in the chicken “factories” that have to produce millions of livestock animals weekly to meet the constantly increasing demands for human food.

The growth promotant (repartitioning) activity of adrenergic beta-receptor agonists has been demonstrated in various livestock species (Fawcett, J. P. et al. 2004; Watkins L. E. et al. 1990 and Mills S. E., 2001, which publications are hereby included in their entirety by reference.) Thus, the growth of livestock animals can be accelerated and the leanliness can be improved by the use of repartitioning adrenergic beta-receptor agonists (Aberg et al., U.S. Pat. No. 6,110,974).

Bacterial infections have been treated or prevented with the admixture of antibiotics into the animal feed, but this praxis is being banned in an increasing number of countries due to the risk for the development of antibiotic-resistant strains of bacteria. Furthermore, antibiotics have effects only on bacterial infections, not on other infections. An alternative to the use of antibiotics may be to strengthen the immune system in the livestock animals, which may be achieved by the administration of immune-modulating compounds, such as for example by the administration of beta-glucans to the animals. Beta-glucans are naturally occurring polysaccharides that are usually isolated from biological materials such as for example from brewer's or baker's yeast, fungi, or bacterial cell walls, according to methods which are well known to those skilled in the art, and described in US Patent Application 20050020490, which is hereby incorporated in its entirety by reference.

Problems in the livestock industry remain, as the repartitioning efficacy of adrenergic beta-receptor agonists decrease over a time period of a couple of weeks and said adrenergic beta-agonists may induce stress and increased mortality in livestock animals.

Stress in livestock animals is a serious condition and may induce poor meat quality, such as for example the PSE syndrome in the swine (pale, soft and exudative meat that becomes dry upon cooking). Stress-induced tachycardia is a serious side-effect of treatment with adrenergic beta-receptor agonists and may lead to cardiac tachyarrhythmias and increased lethality of livestock animals by sudden cardiac death.

### SUMMARY OF THE INVENTION

It has now surprisingly been found that immunomodulating compounds, such as the polysaccharides called beta-glucans, enhance the effects of adrenergic beta-receptor agonists, such as for example ractopamine or salbutamol and the eutomeric enantiomers thereof. A synergistic effect of co-administration of an adrenergic beta-receptor agonist and an immunomodulating beta-glucan has been found, as said beta-glucan will decrease certain side effects of the adrenergic beta-receptor agonist.

An important effect of adrenergic beta-receptor agonists is the repartitioning effect, which means that a decrease of fatty tissues (fat) in combination with an increase of muscular tissues (lean meat) are obtained. The decrease in fatty tissues is mainly achieved by adrenergic beta-receptor agonists activating adrenergic beta-receptors on adipocytes, thereby inducing lipolysis in said adipocytes (fat cells) by activation of triglyceride lipase, through mechanisms that are well known to those skilled in the art of pharmacology and as described in textbooks in pharmacology, such as Goodman & Gilman, 9th Edition, page 208. Beta 1.3/1.6 D-glucan has now surprisingly been found to improve the activity of adrenergic beta-receptor agonist in animals, possibly by causing improved beta-receptor availability, but other mechanisms, such as for example improved signaling cannot be excluded. An important effect of beta-glucans is the remarkable immunomodulating activity of these compounds. These compounds are polysaccharides are potent activators of macrophages and during evolution, specific receptors on various cells, such as macrophages, have been developed. Activated macrophages are of importance for the immunological reactions of all known mammals and in non-mammals, such as birds, fish and crustaceans. Activated macrophages combat infections of various types, such as for example bacterial, viral, fungal and parasitic infections, and activated macrophages will also produce numerous cytokines that will stimulate the immune system in general and boost bone marrow production. Treatment of livestock animals with beta-glucans has been shown to decrease morbidity and mortality, particularly in livestock animals.

The combination of an adrenergic beta-receptor agonist and an immuno-activating compound will be useful in livestock animals, where both the growth promotion by the beta-receptor agonist and the immunological defense by the beta-glucan will be improved by the coadministration of the adrenergic beta-receptor agonist and beta-glucan.

The present invention further comprises the presentation of this combination in an oral formulation to be used as an admixture to the feed of livestock animals or a combination of admixture of the beta-glucan to the feed and admixture of the adrenergic beta-agonist to the drinking water. Alternatively, the beta-glucan can be administered orally in combination with an adrenergic beta-agonist that is administered by inhalation. The financial returns for the breeder using the present invention will be improved.

Formulations containing beta glucans have been described by Sorgente et al. in US 20050020490, which patent application is hereby included in its entirety by reference. Use of beta-glucans in human medicine have been described by Sorgente et al. in U.S. Pat. No. 7,018,986, which is hereby incorporated in its entirety by reference.

Synergism between beta-glucans and antibiotics have been described by Kaiser et al., 1998, which publication is hereby incorporated in its entirety by reference.

The term “eutomeric enantiomer” refers to therapeutically active enantiomers. While the salbutamol molecule has only one chiral center, the ractopamine molecule has two chiral centers. Salbutamol has two optically active isomers, called R-salbutamol and S-salbutamol, and ractopamine has four optically active isomers, called RR-, SS, RS, and SR-ractopamine.

The terms “livestock” or “livestock animal(s)” as used herein refer to animals that are bred for human food, in particular farm animals such as ruminants (as for example cattle, goats and sheep), horses, swine, and deer, birds (such as for example chickens, turkeys, ducks, quails and geese), and farmed fish and farmed crustaceans.

The term “farmed fish” as used herein includes all fish that are farmed and intended for human consumption, for ornamental fish or for fry or fingerlings that are subsequently released in the wild. Examples of farmed fish are farmed barramundi, farmed bluegill, farmed carp species, farmed catfish, farmed cod, farmed goldfish, farmed gourami, farmed perch, farmed salmon, farmed sturgeon, farmed tilapia and farmed trout.

The terms “crustaceans” and “farmed crustaceans” as used herein refer to farmed crustaceans and farmed mollusks and include for example farmed abalone, farmed blue mussel, farmed freshwater crayfish, farmed oyster, farmed prawns, farmed scallop, farmed shrimp, and farmed lobster.

The term “ractopamine” in this document refers to the free amine or to a salt or solvates of RR/SS/RS/SR-ractopamine. Thus, the term “ractopamine” refers to a mixture of approximately equal concentrations of the four enantiomers of ractopamine.

The terms “growth promoter” and “repartitioning agent” and the like, as used herein, refer to a chemical entity that upon administration to livestock animals will have a favorable effect on feed efficiency and on the muscle-to-fat ratio in the carcass of said livestock animals. A growth promoter may, or may not, cause an increase in body weight.

The term “feed efficiency” as used herein, refers to the relationship between feed intake and muscle weight gain in livestock animals. Improved feed efficiency means that the ratio feed intake/muscle weight gain is decreased. Improved feed efficiency also means that the ratio muscle weight gain/feed intake is increased. The term feed efficiency may also refer to the feed intake/weight gain or weight gain/feed intake.

The term “muscle-to-fat ratio” as used herein, refers to the total weight of muscle (meat), divided by the total weight of body fat. The compounds of the present invention cause an increase in muscle weight and a decrease in total body fat, as described elsewhere in this document.

The terms “obese dogs” and “obese cats” are herein defined as dogs or cats being 20% or more overweight as compared with the normal weight of animals of the same species, strain and age. An “overweight” dog or cat is herein refined as an animal being at least 10% heavier than the normal weight of animals of the same species, strain and age.

The term “immunomodulation” as used herein, refers to an improvement of the body's immune system, caused by agents that activate or suppress its functions.

### The Drugs

There are different types of glucans and beta-glucans can exist in various isomeric forms, which are all included in the present invention. Examples of various types of beta-glucans and background information about beta-glucans is available on the Internet, for example an article called “Beta-glucan” that can be found at http://en.wikipedia.org/wiki/Beta-glucan, which article is hereby incorporated in its entirety by reference. D-isomeric forms of beta-glucans are preferred since they are biologically the most active isomers. An insoluble beta 1.3/1.6 D-glucan, most often derived from baker's yeast or brewer's yeast, has a different molecular structure than that of soluble beta glucans and said beta 1.3/1.6 D-glucan has more potent biological activity than most soluble beta-glucans, which is believed to be due to the structural “branching” of this specific beta-glucan. Beta 1.3/1.6 D-glucan is notable for its potent immunomodulating function, although other beta-glucans to varying degree also share this property. Beta glucans are natural polysaccharides occurring for example in the bran of cereal grains, in yeast, and in certain mushrooms. The cereal based beta glucans occur most abundantly in barley and oats and to a lesser degree in rye and wheat. A common source of beta 1.3/1.6 D-glucan is the cell wall of yeast (Saccharomyces cerevisiae). Immunomodulating beta-glucans also exist in the roots of the Chinese plant astragalus (Astragalus membranaceous, Huang Qi), of which extracts are available in health stores in the USA and elsewhere. Said extracts are most often called “Astragalus” and often contain beta-glucans from the roots of Astragalus membranaceous mixed with beta glucans from other sources, such as for example mushrooms. Other sources for beta glucans include some types of seaweed, and various species of mushrooms such as Reishi, Shiitake, and Maitake. Purified beta glucans are commercially available on the open market and beta 1.3/1.6 D-glucan can be purchased for example from BioAgra LLC, Hinesville, Ga., USA, or Immudyne Inc., Florence, Ky., USA or Stirling Products, Perth, Australia.

Beta-glucans have affinity for several pathogen-recognizing receptors, the most important is currently believed to be the Dectin-1 receptor, which is prevalent on splenocytes and has been shown to be the major beta-glucan receptor on macrophages (Reid et al., 2004.) Some preparations of beta-glucans may contain small quantifies of polymannose (“mannan”), which is a polysaccharide with some biological activity, which is insignificant when compared to the activity of beta glucan, particularly since mannan is prone to degradation by glucanases and since no improvement of the activity of beta-glucans was obtained by mannan (Goldman R., 1988).

Sympathomimetic drugs are substances that mimic the effects of the endogenous hormone/neurotransmitter substances norepinephrine (noradrenaline) and epinephrine (adrenaline). Sympathomimetic drugs, such as for example salbutamol, terbutaline, salmeterol, formoterol, ractopamine, zilpaterol and cimaterol are small molecules that activate adrenergic receptors, such as adrenergic beta-receptors in the bodies of humans and animals. These drugs are often referred to as adrenergic beta-receptor agonists, adrenergic beta-receptor activators, beta agonists, beta-stimulators and the like. Drugs such as salbutamol, terbutaline, salmeterol and formoterol and eutomeric isomers thereof are used as bronchodilators in humans and animals, suffering from bronchoconstriction. Adrenergic beta-agonists and eutomeric isomers thereof are also used as growth promoters in livestock animals (U.S. Pat. No. 6,110,974), since said compounds have potent re-partitioning activity, meaning that treated animals develop less fat tissues and more muscle tissues. Thus, ractopamine and zilpaterol are commercially used as growth promotants for livestock animals. Adrenergic beta-receptor agonist drugs may also be used as weight-loss medication since these compounds have potent lipolytic activity, which is sometimes coupled with CNS-mediated appetite-suppressing activity of said drugs. Adrenergic beta-receptor agonist drugs are available in pharmacies upon prescription from a medical doctor or a veterinarian.

Antibiotics are being used both in humans with bacterial infections and in livestock animals to prevent and decrease morbidity and mortality. The effects of antibiotics in livestock animals can be enhanced by co-administration of an antibiotic and an immuno-modulator, such as beta 1.3/1.6 D-glucans. In addition to improved effects on microorganisms, decreased morbidity and decreased mortality, combinations of antibiotics and immuno-modulators will further decrease the use of antibiotics, thereby further decreasing the risk for the development of drug-resistant bacterial strains. The full or partial replacement of antibiotics with beta-glucans will be advantageous from an ecological point of view since the pollution of man-made antibiotics will be fully or in part replaced with environmentally friendly and naturally occurring immunomodulators, such as beta-glucans. There are also financial aspects, since beta-glucan can be cost-effectively extracted from brewers yeast that is readily available commercially.

### Current Treatment

Presently, ractopamine is used as a repartitioning agent for livestock (cattle, swine), zilpaterol is being introduced as repartitioning agent for livestock in the US and R-salbutamol is in development outside the US as a repartitioning agent for livestock, including chicken.

Presently, large amounts of antibiotics are used in livestock animals to prevent morbidity and mortality. However, the use of antibiotics in livestock animals is restricted or banned in most countries, which is due to the risk for development of resistant bacterial strains.

In horses suffering from heaves, adrenergic beta-receptor agonists are used to obtain relief from obstructed airways and antibiotics are used to treat infections, including pulmonary bacterial infections.

### DETAILED DESCRIPTION

It has now surprisingly been found that sympathomimetic compounds, such as for example adrenergic receptor agonists, such as for example ractopamine and R-salbutamol, cause an enhancement of the immunomodulating activity of beta-glucans and in particular beta 1.3/1.6 D-glucan.

The present invention relates to the administration to livestock animals of a combination of an immunomodulating compound, such as a beta glucan compound, preferably beta 1.3/1.6 D-glucan and an adrenergic beta-receptor agonist, such as for example ractopamine, zilpaterol and salbutamol. Beta-glucans, such as beta 1.3/1.6 D-glucan will modulate the immune system and thereby decrease morbidity and mortality. Beta-glucans and particularly beta 1.3/1.6 D-glucan have now been found to inhibit the often fatal effects of stress in animals (Example 4), that may be induced by adrenergic beta-receptor agonists in livestock animals. The explanation for this synergistic activity is not obvious, but a possible potentiation of the immunomodulating activity of beta-glucans by adrenergic beta-receptor stimulation has been described (Elenkov et al., 2000).

The amount of beta glucans to be used in livestock animals to induce immuno-modulation varies widely and depends on the purity and the potency of the beta glucan used, the species of the animals and the effects sought. Administration of beta glucans to livestock animals is usually by oral administration and the beta-glucans are usually mixed with animal feed or nutritive supplement, both of which are typically protein-based. The food composition of the present invention is not particularly limited, and will depend upon the species of the animal consuming the feed. Generally, the food composition is a protein-containing food, having blended therein one or more of the beta-glucans. The food composition may also contain fat, sugars, vitamins or other nutritionally valuable ingredients. The amount of the beta 1.3/1.6-D-glucans needed to induce immuno-modulation in livestock animals is in the range of 1 ppm to 500 ppm in the feed or the drinking water of said animals. A useful dose of beta 1.3/1.6 D-glucans to poultry is in the range of 10 ppm to 100 ppm in the feed, in particular concentrations of about 20 ppm in the feed. A useful concentration of beta 1.3/1.6 D-glucans to swine is in the range of 20 ppm to 400 ppm in the feed, in particular concentrations of about 80-100 ppm in the feed. A useful concentration of beta 1.3/1.6 D-glucans to horses is in the range of 25 ppm to 300 ppm in the feed, preferably about 60-100 ppm. A useful concentration of beta 1.3/1.6 D-glucans to cattle is in the range of 20 ppm to 400 ppm in the feed, often about 60-100 ppm. A useful concentration of beta 1.3/1.6 D-glucans to sheep is in the range of 20 ppm to 300 ppm in the feed, preferably about 60-100 ppm. The dose of beta-glucans to fish is usually included in the feed, which will contain 5 to 500 ppm of the beta-glucan, preferably about 20 to 100 ppm. The dose of beta-glucans to crustaceans is preferentially included in the feed that may contain 2 to 500 ppm of beta 1.3/1.6 D-glucan, preferably from about 10 to about 100 ppm. The treatment of livestock animals with beta-glucans will most likely be determined by regulatory authorities and the treatment period and doses, concentrations and dosing periods are expected to become strictly regulated in most countries. All such doses, drug concentrations and dosing periods of beta-glucans are included in the present invention. The dosing frequency of all drugs is determined by the feeding frequency when the drugs are mixed into the feed and is usually ad lib, since livestock animals most often have continuous access to feed. Likewise, when the drugs are added to the drinking water, the dosing frequency is ad lib, since livestock animals have continuous access to drinking water.

Treatment of livestock animals with beta glucans can be started as soon as possible after the birth of the animal and can continue for the duration of the life of the livestock animals.

It has now surprisingly been determined that immunomodulating compounds, such as beta-glucans and in particular beta 1.3/1.6 D-glucan, cause an enhancement of the effects of sympathomimetic compounds, such as for example ractopamine and R-salbutamol. The mechanism(s) for this potentiation is/are largely unknown, but it is possible that a contributing factor for this potentiation may the surprising finding that beta 1.3/1.6 D-glucan can decrease or inhibit the downregulation of adrenergic beta-receptors. It has previously been described that immune responses may cause an up- or a down-regulation of beta-2 adrenoceptor mRNA in the male rat thymus (Morale, 1992), but we are not aware of any previous study describing effects of beta 1.3/1.6 D-glucan, or any other beta-glucan, on the density of adrenergic beta-receptors. Repeated use of adrenergic beta-2 agonists is known to result in receptor downregulation, which in turn results in decreased effects of the treatment. However, since beta 1.3/1.6 D-glucan has now been found to inhibit said downregulation, it may now be possible to prolong the treatment with a beta-receptor agonist without deteriorating functional activity.

Adrenergic beta-agonists may be administered to live-stock animals orally or parenterally, by injections, infusions or any of a wide array of devices. The administration to livestock animals is usually by the oral route and the beta-agonists are usually mixed with the animal feed, although said compounds may also be mixed into the drinking water. Devices carrying an adrenergic beta-receptor agonist and/or a beta-glucan can be implanted subcutaneously or into the rumen of cattle or sheep in order to release in a predetermined manner, a beta-receptor agonist, a beta-glucan or both a beta-receptor agonist and a beta-glucan.

The amount of an adrenergic beta-agonist to be used in livestock animals to induce repartitioning varies widely and is depending on the potency and the pharmacokinetic properties of the adrenergic beta-receptor agonist in the selected species and the effects sought. Thus, the beta-agonist R-salbutamol is about twice as potent as RS-salbutamol. Salbutamol and ractopamine have approximately the same affinity for adrenergic beta-2 receptors, but salbutamol has higher oral bioavailability than ractopamine and the doses of salbutamol may be lower than the doses of ractopamine, when given to livestock animals. However, the recommended doses for both salbutamol and ractopamine are within the ranges for beta-receptor agonists, given below. In general, the amount used of adrenergic beta-receptor agonist are in the range from about 1 ppm to about 100 ppm of the feed to livestock animals. A useful dose of R-salbutamol to poultry is in the range of 2 ppm to 20 ppm in the feed, most useful about 5-10 ppm. A common dose of R-salbutamol to swine is in the range of 5 ppm to 100 ppm in the feed, often about 10-20 ppm. A common dose of ractopamine to swine is approximately 20 ppm in the feed. A useful concentration of R-salbutamol to horses is in the range of 2 ppm to 60 ppm in the feed, often about 2-20 ppm. R-salbutamol can also be used in horses, suffering from heaves and the adrenergic beta-receptor agonist may then be administered by an inhaler at a dose to be decided by the caring veterinarian. A useful concentration of R-salbutamol to cattle is in the range of 5 ppm to 100 ppm in the feed, often about 10-40 ppm. A useful concentration of ractopamine in the feed for cattle is usually 10 to 30 ppm or a total dose of about 200 mg/steer/day. A useful concentration of zilpaterol in the feed for cattle is usually 5 to 10 ppm or a total dose of 60 to 90 mg zilpaterol/steer/day. A useful concentration of R-salbutamol to sheep is in the range of 5 ppm to 100 ppm in the feed, often about 10-40 ppm. A useful concentration of R-salbutamol in the feed to farmed fish is in the range of 5 ppm to 100 ppm, often about 10-60 ppm. R-salbutamol is relatively toxic to crustaceans and LD50 for racemic salbutamol in barnacle naupii is approximately 1.8 μg/ml and the concentrations used to treat crustaceans have to be selected accordingly (Samuel, O. H. Y et al., 2003).

The treatment period of livestock animals with adrenergic beta-receptor agonists varies between species. Treatment of poultry with adrenergic beta-agonists can be started when adrenergic beta-receptors become fully expressed in the animals, usually at 2 or 3 weeks of age and the poultry are treated continuously up to or including the day of slaughter. The treatment period of swine with adrenergic beta-receptor agonists is usually the last 2 to 4 weeks before slaughter and the treatment period of cattle is usually the last 4 to 6 weeks before slaughter. The allowed treatment periods of livestock animals with adrenergic beta-receptor agonists is usually determined by regulatory authorities and the treatment period and doses, drug concentrations and dosing periods are strictly regulated in most countries. All such doses, drug concentrations and dosing periods of adrenergic beta-receptor agonists are included in the present invention. The treatment of livestock animals with non-halogenated adrenergic beta-receptor agonist can most often continue up to and even include the day of slaughter, while the treatment of livestock animals with long-acting adrenergic beta-receptor agonists may have to be stopped about two days before slaughter in order to limit the concentrations of the repartitioning compounds in the carcasses of said livestock animals an in order to limit the exposures of humans, eating food comprising parts from said carcasses.

The dosing period for the combination of an adrenergic beta-receptor agonist and an immunomodulating compound will coincide with the dosing period for the adrenergic beta-receptor agonist as stated above. The doses of the adrenergic beta-receptor agonists and the beta-glucan during co-administration will be in the ranges as indicated above.

In another embodiment of the present invention, unexpected effects on the incidence of liver abscesses are seen after administration of a combination of an adrenergic beta-receptor agonist and beta 1.3/1.6 D-glucan to ruminants. As a background, the incidence of liver abscesses in most cattle feedlots averages from 12 to 32 percent. Severe liver abscesses are associated with decreased feed intake and decreased average daily gain. Abscesses that exist during the feeding period affect performance, whereas abscesses that exist at slaughter affect yield and condemnation rates. Liver abscesses are often the result from switching cattle too quickly to high-energy concentrate diets, often in combination with the use of a repartitioning adrenergic beta-receptor agonist. This may cause acute rumenitis by allowing the rumen microflora to produce lactic acid that leads to ulcerative lesions of the rumen mucosa, which in turns allow entry of bacteria into the bloodstream where they are carried to the liver, where colonies of bacteria, mainly Fusobacterium necrophorum and Actinomyces pyogenes, grow, which results in local infections, necrosis and liver abscesses (U.S. Pat. No. 7,207,289, which is hereby included in its entirety by reference). Traditionally, macrolide antibiotics have been used to reduce the incidence of liver abscesses. Alternatively, a polyether ionophore antibiotics such as momensin can be used.

It has now surprisingly been found that beta-glucans, such as beta 1.3/1.6 D-glucan, can replace said antibiotics. Through unknown mechanism said beta-glucans will cause unexpected reduction or total elimination of liver abscesses in ruminants.

A beta 1.3/1.6 beta glucan can also decrease or inhibit the development of liver abscesses in ruminants being administered an adrenergic beta-receptor agonist.

The mechanism(s) for this unexpected effect of said therapy is/are unknown, but since it has now been found that beta 1.3/1.6 D-glucan is unexpectedly stable in the rumen (Example 1), it can be speculated that direct effects of beta-glucan against injury on the rumen mucosa may be involved. It is also possible that immunological effects may be involved in causing an unexpected reduction in liver abscesses by beta 1.3/1.6 D-glucan.

The present invention is also particularly useful in obese companion animals such as overweight or obese cats and overweight or obese dogs, being treated with adrenergic beta-agonists to achieve loss of weight. Both ractopamine and salbutamol will stimulate adrenergic beta-1 and beta-2 receptors and since the adipocytes of said animals have both types of receptors, lipolysis in these species may be mediated by both adrenergic beta-1 and beta-2 receptors (Harms et al., 1982; incorporated by reference.) Continued dosing with an adrenergic beta-receptor agonist to obese companion animals is initially causing significant weight loss, which mainly is believed to be due to the lipolytic activity of the drug. However, weight loss by an adrenergic beta-receptor agonist is rapidly decreasing over time and usually disappears within a few weeks, which most likely is due to decreased lipolytic activity that may be caused by down-regulation of adrenergic beta-receptors, probably on the adipocytes. As mentioned above, studies in laboratory animals surprisingly demonstrate that continued co-administration of beta 1.3/1.6 D-glucan with an adrenergic beta-receptor agonist results in an increased population of adrenergic beta-receptors in tissues of said laboratory animals. This may result in a sustained lipolytic effect of adrenergic beta-receptor agonists, when combined with beta 1.3/1.6 D-glucan. It can therefore be concluded that weight-loss efficacy by continued treatment with an adrenergic beta-receptor agonist can be sustained when said treatment is combined with continued treatment with an effective dose of a pure beta 1.3/1.6 D-glucan, such as Agrastim® (BioAgra LLC, Hinesville, Ga., USA) that is used in all studies performed by us.

The present invention is also particularly useful in horses suffering from heaves since continued administration of a beta 1.3/1.6 D-beta glucan inhibits or decreases adrenergic beta-receptor downregulation that is known to occur after repeated stimulation with beta-receptor agonists, such as for example R-salbutamol or ractopamine. Those skilled in the art of veterinary medicine realize that it is of major importance to sustain the bronchodilating activity of a beta-2 receptor agonist in horses that are suffering from heaves, which is a chronic disease. Additionally, horses suffering from heaves very often have respiratory/pulmonary infections (Theegarten et al., 2008.) Such pulmonary infections in horses suffering from heaves are not always bacterial infections, but may also be of viral, fungal, mycoplasmic or parasitic origin and may therefore not be controlled by antibiotics. However, beta 1.3/1.6 D-beta glucans will also improve the immune defense against all infections, making the use of a combination of a beta-receptor agonist and a beta-glucan exceptionally valuable in horses suffering from heaves.

Since beta-glucans have shown activity against cancer (Morikawa et al. 1985), administration of immune modulating beta-glucans together with an adrenergic beta-receptor agonist can have anti-carcinogenic effects and therefore decrease the risk for the development of various types of cancer, such as for example uterine leiomyomas, which is a type of uterus cancer that is known from animal studies to be induced by adrenergic beta-receptor agonists (Gopinath et al., 1987.)

Breeders of livestock animals, administrating a combination of an adrenergic beta-receptor agonist and a beta 1.3/1.6 D-glucan to said livestock animals, will particularly benefit from administering the herein described combination therapy to the animals. Thus, the method of the present invention yields leaner animals, which command higher prices from the meat industry. The livestock animal breeder will appreciate that feed efficiency, repartitioning and animal growth rate are significantly enhanced when the methods of the present invention are followed. The breeders of livestock animals will also benefit from administering a combination of a beta 1.3/1.6 D-glucan and a beta-receptor agonist, since stress-induced side effect of the adrenergic beta-agonist will be decreased.

### Biological Effects

### BACKGROUND

To those skilled in the art of pharmacology, it is known that synthetic adrenergic beta-receptor agonists have numerous effects, that are similar to the effects of endogenous adrenergic beta-receptor agonists, of which adrenaline and noradrenaline are the most well known.

Three types of adrenergic beta-receptors have been described: Stimulation of beta-1 receptors leads—for example—to increased heart rate and increased cardiac contractility. Stimulation of adrenergic beta-2 receptors leads—for example—to relaxation of various types of smooth muscles, and to increased lipolysis. Adrenergic beta-3-adrenergic receptors are—for example—involved in the regulation of thermogenesis.

Beta glucans of the beta 1.3/1.6 D-glucan type activate and modulate the immune system in both humans and animals, including fish, birds and crustaceans, thereby making both humans and animals more resistant to bacterial, viral, fungal and parasitic infections and also to tumor growth. Thus, the complement receptor 3 on polymorphonuclear leucocytes (PMNs) has two distinct binding sites, one of which, the CR3 receptor, has a domain that binds to polysaccharides or fractions thereof.

There have been reasons to believe that beta 1.3/1.6 D-glucan may undergo pre-systemic metabolism in the rumen of cattle. It has therefore been believed that administration of beta 1.3/1.6 D-glucans may have no or very limited therapeutic effects in cattle and other ruminants. However, results from ongoing studies demonstrate that beta 1.3/1.6 D-glucan isolated from brewers yeast, (Agrastim®, BioAgra LLC, Hinesville, Ga., USA), which is a particularly pure form of beta 1.3/1.6 D-glucan, undergoes insignificant pre-systemic metabolism in cattle and will therefore be of significant therapeutic value in this species. Thus, beta 1.3/1.6 D-glucan, as well as combinations thereof with adrenergic beta-agonists can be used in cattle.

### EXAMPLES

### Example 1

### Presystemic Stability of a Beta Glucan in Rumen of Cattle

Since the rumen of cattle is particularly rich in glucanases, there have been reasons to believe that beta 1.3/1.6 D-glucan may undergo pre-systemic metabolism in cattle. Thus, it has therefore been believed by those knowledgeable in beta-glucans that administration of beta 1.3/1.6 D-glucans and other beta-glucans may have none or very limited therapeutic effects in cattle and possibly also in other ruminants.

A. Agrastim® (beta 1.3/1.6 D-glucan, Bioagra, Hinesville, Ga., USA) was incubated with rumen fluid from cattle. Rumen fluid contains an array of enzymes, derived from the feed, the rumen walls and bacteria. Glucanases are present in the rumen to digest fibers and it has therefore been assumed that beta 1.3/1.6 D-glucan might be highly instable in the rumen of cattle. The initial study was performed in vitro to investigate the stability of a beta glucan in rumen fluid. Since the beta 1.3/1.6 D-glucan was the only dry, non-soluble material in the incubates during the present study, the disappearance of dry matter was equal with the degradation of said beta glucan in the rumen fluid.

Test results are shown in the following table:

| Incubation Period (hrs) | Remaining beta-glucan (%) |
|---|---|
| 0.5 | 98 |
| 2.0 | 97 |
| 4.0 | 85 |
| 8.0 | 60 |
| 12.0 | 20 |

The results demonstrate that Agrastim® (beta 1.3/1.6 D-glucan, BioAgra, Hinesville, Ga., USA) was degraded to not more than 15 percent and to not more than 3 percent after incubation in rumen fluid from cattle for 4 hours and 2 hours, respectively.

B. In an in vivo follow-up study in cattle, it has now been determined that the same beta glucan (Agrastim® BioAgra) clears the rumen of cattle within 2 hours.

It can be concluded from these two studies that orally administered beta 1.3/1.6 D-glucan will undergo insignificant presystemic metabolism in the rumen and that this beta-glucan will clear the rumen in cattle, whereupon said beta-glucan may be absorbed from the intestine.

To our knowledge, this is the first laboratory evidence, demonstrating that beta 1.3/1.6 D-glucan is not presystemically metabolized in cattle.

### Example 2

### In Vivo Study in Cattle

The purpose of this study was to determine if beta 1.3/1.6 D-glucan will enhance the immune system after oral administration to ruminants. The present study was performed at a farm with a high rate of mortality of newborn calves from Cryptosporidiosis and upper respiratory infections.

Two groups of 13 newborn calves were studied. All animals were given milk replacement and beta 1.3/1.6 D-glucan was added to the milk replacement for the second group. Thus, the animals of the second treatment group of 13 calves were administered Agrastim® at a dose of 500 mg on the first day and 250 mg/day for the following 8 weeks.

Test results are shown in the table below:

| | Calves | Calves |
|---|---|---|
| Treatment | Born | Weaned |
| Milk Replacement | 13 | 7 |
| Milk Replacement + Agrastim ® | 13 | 12 |
| 500 mg-day 1 | | |
| 250 mg-day 2-56 | | |

The results demonstrate that survival was improved from 7 out of 13 calves to 12 out of 13 calves by treatment with beta 1.3/1.6 D-glucan (Agrastim®, BioAgra LLC, Hinesville, Ga., USA) and it can be concluded that this beta-glucan is absorbed in cattle and will significantly improve the immune system in cattle. Conclusion: To our knowledge, this is the first hard evidence demonstrating that beta 1.3/1.6 D-glucan has therapeutic activity in cattle.

### 3. Studies on Down-Regulation of Adrenergic Beta-2 Receptors

The density of adrenergic beta-2 receptors in animals undergoing immuno-modulation by continuous treatment with beta 1.3/1.6 D glucan are investigated, using standard receptor binding methodology. The populations of adrenergic beta-2 receptors on cells (adipocytes) from rats are studied. Results demonstrate that the adrenergic beta-2 receptors were down-regulated after one and two weeks of continuous administration of an adrenergic beta-receptor agonist (salbutamol) by subcutaneously implanted Alza osmotic pumps. Results also show that said down-regulation surprisingly is decreased or inhibited in animals that are administered a beta 1.3/1.6 D-glucan (Agrastim®, BioAgra, Hinesville, Ga., USA) for one week before the implantation of the ALZA pumps and continuously thereafter.

It is concluded that beta 1.3/1.6 D-glucan has the ability to inhibit the down-regulation of adrenergic beta-2 receptors, whereby therapeutic activities of adrenergic beta-2 receptor agonists, such as for example repartitioning, weight loss or bronchial dilatation, will be significantly improved.

### 4. Studies on Stress-Induced Mortality in Mice.

A modification of the physostigmine-induced lethality test that successfully was used for testing of sedation by Schering in the loratadine project (Villani F. J., et al., U.S. Pat. No. 4,659,716, 1987) and by Sepracor in the desloratadine project (Aberg, G. et al. U.S. Pat. No. 5,595,997) was used. In short, mice that previously had not been housed together, suffer from severe stress when placed together (n=10) in a new environment, such as a small cage with which they are not familiar. The stress is evidenced by significantly increased spontaneous motor activity by the animals and the stress is further increased by injections of physostigmine (1.0 mg/kg s.c.), which produces nearly 100% lethality. The adrenergic beta-receptor agonist ractopamine increased the stress in said laboratory animals and caused increased mortality in animals that had been given a sub-lethal dose of physostigmine. Similarly, increased stress by ractopamine in livestock animals has been reported by Marchant-Forde et al., 2003. In the present study, some groups of mice were pretreated by oral administration of beta 1.3/1.6 D-glucan daily for five days, while other groups were not pretreated with said beta-glucan. The number of surviving mice was counted 30 minutes after the animals had been placed in the test-cage, which was 60 min after the administration of ractopamine.

The test results unexpectedly demonstrate significantly decreased stress and stress-induced mortality in ractopamine-treated animals when said animals had been pretreated with beta 1.3/1.6 D-glucan.

It is concluded that beta 1.3/1.6 D-glucan unexpectedly decreased the stress induced by the adrenergic beta-receptor agonist. Thus, when translated into livestock animals, ractopamine will—as expected—exert a repartitioning effect and beta 1.3/1.6 D-glucan will—as expected—decrease the morbidity and mortality that is caused by infections. However, it has now been found that in addition to said expected and beneficial drug effects, the present test results point to a remarkable and unexpected synergism between the drugs, since pretreatment with a beta-glucan inhibited drug-induced side effects of stress and the increased mortality that otherwise was induced by treatment of the animals with ractopamine.

### EQUIVALENTS

Any adrenergic beta-2 receptor agonist(s), including but not limited to ractopamine, salbutamol, zilpaterol and cimaterol, may be used in combination with any beta-glucan(s) according to the present invention.

1. A method of decreasing drug-induced side effects in a livestock animal, comprising administering an effective amount of a beta 1.3/1.6 D-glucan to an animal being treated with a repartitioning dose of an adrenergic beta-receptor agonist.

2. A method of improving repartitioning a livestock animal, comprising administering to said animal an effective amount of a beta glucan in combination with an adrenergic beta-receptor agonist.

3. A method of reducing the incidence of liver abscesses in cattle, comprising administering to cattle an effective amount of a beta glucan.

4. The method of claim 3, wherein said beta glucan is co-administered with an adrenergic beta-receptor agonist.

5. The method of claim 1, 2 or 4, wherein said beta-receptor agonist is selected from the group consisting of ractopamine, salbutamol, zilpaterol, cimaterol and eutomeric isomers thereof.

6. The method of claim 1, 2, 3 or 4, wherein said beta glucan is beta 1.3/1.6 D-glucan.

7. The method of claim 1, 2, 3 or 4, where said livestock animal is selected from the group consisting of swine, cattle, sheep, horses, chicken, turkeys, farmed fish and farmed crustaceans.

8. The method of claim 1, 2, 3 or 4, where said animal is administered a beta glucan at a concentration ranging from 1 to 500 ppm.

9. The method of claim 1, 2 or 4, where said animal is administered an adrenergic beta-receptor agonist at a concentration ranging from 1 to 100 ppm.

10. The method of claim 1 or 2, where said animal is a swine, administered a beta 1.3/1.6 D-glucan at a concentration ranging from 20 to 400 ppm in combination with an adrenergic beta-2 agonist at a concentration ranging from 5 to 100 ppm.

11. The method of claim 1 or 2, where said animal is a ruminant selected from the group consisting of cattle and sheep, administered a beta 1.3/1.6 D-glucan at a concentration ranging from 20 to 400 ppm in combination with an adrenergic beta-2 agonist at a concentration ranging from 5 to 100 ppm.

12. The method of claim 1 or 2, where said animal is a horse, administered a beta 1.3/1.6 D-glucan at a concentration ranging from 25 to 300 ppm in combination with an adrenergic beta-2 agonist at a concentration ranging from 2 to 60 ppm.

13. The method of claim 1 or 2, where said animal is a bird selected from the group consisting of chicken and turkeys, administered a beta 1.3/1.6 D-glucan at a concentration ranging from 5 to 500 ppm in combination with an adrenergic beta-2 agonist at a concentration ranging from 2 to 20 ppm.

14. The method of claim 1 or 2, where said animal is a farmed fish, administered a beta 1.3/1.6 D-glucan at a concentration ranging from 50 to 500 ppm in combination with an adrenergic beta-2 agonist at a concentration ranging from 5 to 100 ppm.

15. A feed composition for livestock animals comprising the admixture with protein-containing food materials a beta glucan and an adrenergic beta-receptor.
